(19) United States
(12) Patent Application Publication
(10) Pub. No.: US 2025/0186214 A1
(43) Pub. Date: Jun. 12, 2025

(54) INLAY GLENOID IMPLANTS AND METHODS

(71) Applicant: Anika Therapeutics, Inc., Bedford, MA (US)

(72) Inventor: Nicholas Ryan Higdon, Warsaw, IN (US)

(21) Appl. No.: 18/972,380

(22) Filed: Dec. 6, 2024

Related U.S. Application Data
(60) Provisional application No. 63/607,329, filed on Dec. 7, 2023.

(51) Int. Cl. A61F 2/40

(52) U.S. Cl. CPC A61F 2/4081

(57) ABSTRACT

Embodiments described herein relate to total shoulder arthroplasty devices and related systems, methods, and kits useful in implanting glenoid implants in the glenoid fossa of scapula bones. In some embodiments, a glenoid implant device is disclosed including a screw and a liner. In some embodiments, the liner is configured to decouple from the screw during a revision surgery. In some embodiments, the liner has an inner portion configured to couple with a driver to dispose the implant at least partially into a bone.

### CROSS-REFERENCE TO RELATED APPLICATIONS

This application claims the benefit of priority to U.S. Provisional Patent Application Ser. No. 63/607,329, filed Dec. 8, 2023, hereby incorporated by reference in its entirety.

### TECHNICAL FIELD

The present disclosure is related to devices, systems, methods, and kits for total shoulder surgery. Specifically, the disclosure is directed to glenoid implants and related instruments and methods for performing surgery to implant such implants.

### BACKGROUND

Articular cartilage covers the ends of the bones in the human body, particularly where one bone interfaces with another bone such as in a joint. Articular cartilage is a smooth, load bearing, and lubricious tissue which allows one bone to slip past another bone while maintaining strength during movement. When a bone is injured, this articular cartilage may also be damaged. Furthermore, as the body ages articular cartilage can naturally break down, causing one bone to rub against another bone leading to pain for the patient, reduced mobility, and osteoarthritis.

### SUMMARY

In one aspect, embodiments described herein relate to glenoid implants. The glenoid implant may include a liner having a liner inner surface and a liner outer surface. The liner inner surface has a concave profile and is configured to receive a corresponding convex surface of a humeral head or humeral implant. The glenoid implant further includes a screw having a mantle portion and a pedestal portion that extends distally from the mantle portion. The mantle portion is configured to be coupled with a distal portion of the liner outer surface such that the screw extends distally from the liner. The pedestal portion has one or more threads disposed thereabout and configured to secure the implant to a glenoid fossa at an excision site thereof. The glenoid implant is configured to be disposed entirely or substantially entirely within the excision site, thereby having an inlay configuration. In some embodiments one or both of the mantle portion and the pedestal portion comprise a porous material.

In some embodiments, the porous material comprises an open cell porosity structure, a closed cell porosity structure, or a combination thereof. In some embodiments, the liner comprises an inner cavity portion about a distal portion of the liner inner surface. In some embodiments, the inner cavity portion has a generally cylindrical shape. In some embodiments, the inner cavity portion has a generally hexagonal prism shape. In some embodiments, the screw comprises a metal material. In some embodiments, the liner comprises a polyethylene material. In some embodiments, the glenoid implant further comprises an antioxidant material embedded in the polyethylene material. In some embodiments, the glenoid implant further comprises a textured interface disposed between the screw and the liner. In some embodiments, the textured interface has a checkerboard texture. In some embodiments, the glenoid implant further comprises at least one notch disposed in the liner outer surface. In some embodiments, the at least one notch includes a first notch and a second notch disposed in the liner outer surface. In some embodiments, further comprises a plurality of indents disposed in the liner outer surface. In some embodiments, the plurality of indents have a generally concave. In some embodiments, the plurality of indents have a generally convex indent surface. In some embodiments, the plurality of indents is disposed at least partially in the at least one notch. In some embodiments, the plurality of indents are arranged radial-symmetrically about a longitudinal axis of the glenoid implant. In some embodiments, the indents are configured to retain bone cement to adhere the glenoid implant to the glenoid fossa. In some embodiments, the one or more threads comprise double helix threads. In some embodiments, the liner is configured to couple with the mantle portion of the screw through a threaded connection. In some embodiments, the liner is configured to decouple from the mantle portion of the screw through rotation. In some embodiments, one or more threads is configured to secure the implant to a glenoid fossa through rotation therein.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed configuration, the term “substantially” may be substituted with “within [a percentage] of” what is specified, where the percentage includes 0.1, 1, 5, and 10 percent.

Further, an apparatus or system that is configured in a certain way is configured in at least that way, but it can also be configured in other ways than those specifically described.

The terms “comprise” (and any form of comprise, such as “comprises” and “comprising”), “have” (and any form of have, such as “has” and “having”), and “include” (and any form of include, such as “includes” and “including”) are open-ended linking verbs. As a result, an apparatus that “comprises,” “has,” or “includes” one or more elements possesses those one or more elements, but is not limited to possessing only those elements. Likewise, a method that “comprises,” “has,” or “includes” one or more steps possesses those one or more steps, but is not limited to possessing only those one or more steps.

Any configuration of any of the apparatuses, systems, and methods can consist of or consist essentially of—rather than comprise/include/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one configuration may be applied to other configurations, even though not described or illustrated, unless expressly prohibited by this disclosure or the nature of the configurations.

Some details associated with the configurations described above and others are described below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale (unless otherwise noted), meaning the sizes of the depicted elements are accurate relative to each other for at least the configuration depicted in the figures.

FIG. 1 illustrates a glenoid implant, according to a first embodiment herein.

FIG. 2 illustrates a view of the proximal portion of a liner of a glenoid implant with a cylindrical shaped inner portion, according to embodiments herein.

FIG. 3 illustrates a view of the proximal portion of a liner of a glenoid implant with a hexagonal prism shaped inner portion, according to embodiments herein.

FIG. 4 illustrates a glenoid implant, according to a second embodiment herein.

### DETAILED DESCRIPTION

Systems, methods, and devices provided herein relate to a total shoulder arthroplasty (TSA) shoulder surgery. Each of the systems, components, and methods disclosed herein provides one or more advantages over traditional systems, components, and methods. Various embodiments of the TSA devices, systems, components, and methods are disclosed herein.

For example, the shoulder joint comprises bones covered in articular cartilage. The scapula bone has a glenoid region or a glenoid fossa, which is a concave depression in the scapula bone that forms the shoulder joint with humorous bone in humans. The articular epiphysis of each bone is covered in articular cartilage which helps to lubricate, cushion, and stabilize the joint during movement. Injuries to the shoulder joint or degenerative conditions, such as osteoarthritis, can damage the articular cartilage and lead to pain, stiffness, and reduced range of motion in the shoulder for the patient.

In some cases, the extent of damage necessitates repair to the cartilage using one or more implants. For example, a total shoulder arthroplasty (TSA) is a procedure used to place an implant in the each of the articulating surfaces of the glenoid fossa and the humeral head.

A TSA is an example of a total joint replacement which involves removing the upper condyle or a portion of the upper condyle of an articulating bone in addition to a portion of the corresponding articulating surface in the corresponding bone. An implant may then be placed into the bone typically using a stem and cemented into place. When replacing a joint with one or more implants it is important to choose an implant that is mechanically stable and allows for full mobility and movement in the joint. To maintain full mobility and movement in the joint, a sufficient amount of bone must be removed within the joint to allow room for the implants to be placed.

Traditional TSA procedures include impacting implants into bone such as though applying a repetitive downward pressure (e.g., hammering). However, impacting implants into bone may damage surrounding bone resulting in bone loss. Similarly, other techniques for placing implants in TSA procedures include excising a portion of bone. Excising a portion of bone also may result in increased bone loss and increased time for surgery.

However, implant placement and may result in bone loss, which may compromise joint stability, longevity, function, and ability to perform revision surgery. Several factors contribute to bone loss including implant design and the amount of bone that is needed to be removed to place a specific implant design. Implant design can affect the extent of bone loss by altering the amount of bone needed to be removed when placing the implant, load distribution, and stress patterns in the surrounding bone. When placing a new implant or moving an existing implant, the amount of healthy bone available for placement of the implant will impact the ease and speed of placement surgery. Without sufficient healthy bone, an implant cannot be placed. Therefore, there is a need for tools, systems, and methods to preserve as much healthy bone as possible when performing orthopedic surgeries.

While joint replacement surgery is generally successful, some patients may experience complications that require revision surgery. Revision surgery is a more complex and challenging procedure that is performed to remove an existing implant and place a new implant into a joint. Revision surgery may be necessary due to a variety of reasons, such as implant wear and tear, infection, and mechanical failure. In some cases, the original implant may become loose or dislocated, causing pain, instability, and reduced mobility. Implant infections can also occur, and in some cases, antibiotics alone may not be enough to treat the infection. Mechanical failure, such as implant fracture or breakage, can cause severe pain, swelling, and loss of function.

Revision surgery is typically more complex than the initial joint replacement surgery, as it involves removing the original implant, addressing any bone loss or tissue damage that may have occurred, and replacing the implant with a new device. Revision surgery is easier to perform and has a higher chance of success when there is sufficient healthy native bone to attach a new implant. Therefore, there is a need to develop improved methods, systems, and devices for preserving bone when originally placing an implant and for revision surgeries.

Design consideration for an orthopedic implant should include maximizing the patient's comfort, minimize damage to surrounding areas, minimize potential further injury, maximize the functional life of the implant, minimize healthy bone loss, and be easy to install and reinstall in a revision surgery. The systems, methods, and devices described herein are directed towards preserving healthy bone and reducing surgery time for orthopedic surgeries. In some embodiments, the devices, systems, and methods described herein are directed towards a TSA.

As used herein, the terms “proximal” and “distal” refer to the proximal and distal directions relative to a surgeon or other medical professional holding the component or tool. The term “proximal” refers to an area, surface, or point situated closer to the surgeon or other medical professional, and thereby further from a center of patient or subject. The term “distal” refers to an area, surface, or point situated further from the surgeon or other medical professional, and thereby closer to the center of the patient or subject.

Glenoid and/or humoral implants may be placed in a subject (e.g., patient) to add the articulating surfaces of a shoulder joint. Specifically, the glenoid and/or humeral implants are disposed at least partially within the humerus and/or glenoid fossa of the scapula bones. Some embodiments described herein are directed towards devices used for shoulder surgery where an implant may be free floating, uncemented, or mechanically secured into bone.

Disclosed herein, in some aspects, are systems and methods for performing a total shoulder arthroplasty surgery. In some embodiments, the system comprises a humeral implant and/or a glenoid implant. In some embodiments, the glenoid implant has a liner portion and a screw portion configured to be placed at least partially into a glenoid of a patient.

In some embodiments, the glenoid implant is configured to be disposed into and coupled with a bone in the glenoid through a screw mechanism. In some embodiments, such screw mechanism is sufficient to couple and secure the glenoid implant to the glenoid, thereby reducing or eliminating a need to use other coupling mechanisms (e.g., bone cement) for securing the glenoid implant. Inserting the glenoid implant using the screw mechanism may reduce surgery time for the patient as compared with other coupling mechanisms for inserting a glenoid implant (e.g., via bone cement). In some embodiments, the glenoid implant may sit flush with the surrounding bone. For example, in some embodiments, the glenoid implant sits entirely or substantially entirely within an excision site at the glenoid of the patient, thereby having an inlay configuration at the glenoid. One of the benefits of the glenoid implant having an inlay configuration is the resulting reduced amount of corresponding bone needed to be removed as compared to other glenoid implant insertion configurations, thus preserving more healthy bone.

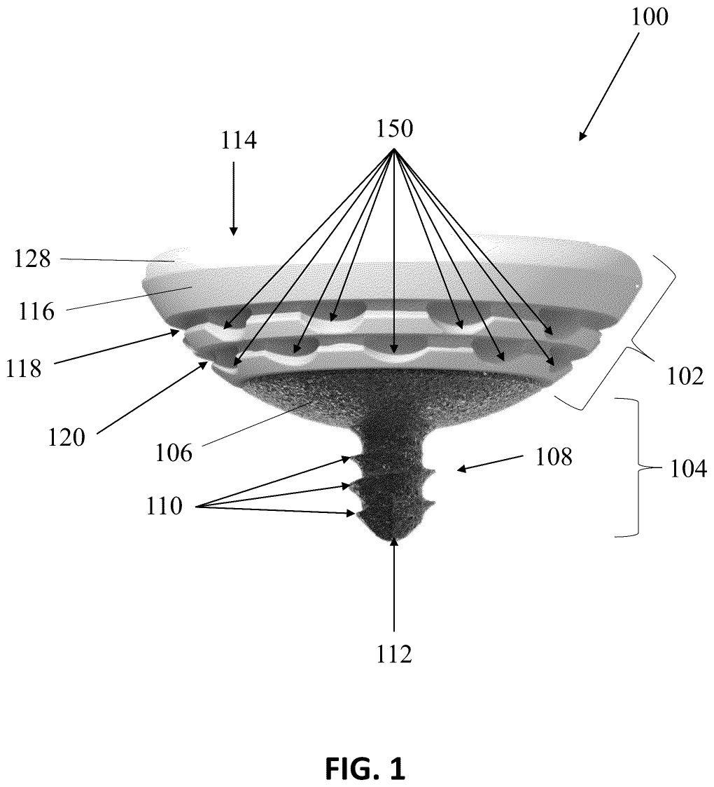

FIG. 1 illustrates an example of an implant 100 having a liner 102 and a screw 104. In some embodiments, the liner 102 and the screw 104 are coupled together. In some embodiments, the liner 102 and the screw 104 are configured as a single component (e.g., a monolithic component). In some embodiments, the liner 102 and the screw 104 are configured as a single component configured to break apart into the liner 102 and the screw 104 when a force is applied.

In some embodiments, the liner 102 and the screw 104 are detachably coupled. In some embodiments, the liner 102 is configured to decouple from the screw 104 by twisting the liner 102 relative to the screw 104. In some embodiments, the liner 102 is configured to decouple from the screw 104 by pulling the liner 104 away from the screw in a direction parallel to the longitudinal axis of the implant 100. In some embodiments, an interface between the screw 104 and the liner 102 has a texture to facilitate decoupling the two components. In some embodiments, the texture is a checkerboard pattern where squares of black represent areas of adhesion at the interface and squares of a white represent areas of no adhesion between the two components at the interface. In some embodiments, the screw portion 104 of the implant 100 is configured to decouple from the liner portion 102 of the implant 100, so as to attach to a new liner (not shown) in a revision surgery. In some embodiments, the screw portion 104 of the implant 100 remains within the bone while the liner 102 is removed and replaced with a new liner, such as during a revision surgery. In some embodiments, the liner portion 102 is pried off of the screw portion 104 while the screw portion 104 is at least partially embedded in a bone so as to decouple the screw portion 104 and the liner portion 102.

In some embodiments, the implant 100 has a substantially circular outer diameter. In some embodiments, the screw 104 is secured to a bone using one or more external threads, ribs, protrusions, bone cement, barbs, grooves, or any other structure that enables the screw 104 to be secured to the bone. In some embodiments, the screw 104 is configured to be drive the implant 100 at least partially into a bone. In some embodiments, the implant 100 is configured to threadably engage with bone via the screw portion 104. For example, in some embodiments, the screw portion 104 has one or more threads disposed thereabout that is configured to be inserted into the glenoid in screw configuration. In some embodiments, the screw portion 104, and thereby implant 100, is configured to be removed by unscrewing the screw portion 104 from the bone. In some embodiments, the implant 100 is secured to the bone via the screw portion 104 being threadably engaged thereto, without using any bone cement, thereby enabling the implant 100 to be configured for such unscrewing.

In some embodiments, the screw 104 has a mantle portion 106 and pedestal portion 108. In some embodiments, the pedestal portion has at least one thread 110 disposed circumferentially around an outer surface of the pedestal 108. In some embodiments, the thread 110 has a single helix configuration. In some embodiments, the thread 110 has a double helix thread configuration. A double helix thread may facilitate fewer rotations to dispose the implant 100 in a bone. Fewer rotations of a screw during surgery may result in a faster surgery. Further, surgeries with shorter times in the operating room reduce the risk of post-operative complications to a patient.

In some embodiments, the mantle portion 106 has a proximal surface configured to couple with at least a portion of the liner 102 (e.g., a distal surface of the liner). In some embodiments, the proximal surface of the mantle portion 106 has a concave configuration for coupling with and/or receiving at least a portion of the liner 102. In some embodiments, pedestal portion 108 extends from a convex, distal surface of the mantle portion 106. In some embodiments, the pedestal portion 108 extends from a center of the mantle portion 106 convex distal surface.

In some embodiments, the screw 104 is made of a porous material. The porous material may have an open-cell porosity structure, a closed-cell porosity structure, or any combination thereof. The porous material may be configured to facilitate bone ingrowth into the screw 104 when the implant 100 is disposed at least partially into a patient's bone. In some embodiments, the screw 104 is implanted into a glenoid region of a scapula bone. In some embodiments, the screw 104 is made of a metal material. In some embodiments, the metal material comprises titanium, cobalt, nickel, chromium, iron, carbon, steel, or alloys thereof, or any combination thereof.

In some embodiments the screw 104 is formed through an additive manufacturing technique. In some embodiments the implant 100 is formed through an additive manufacturing technique. In some embodiments, the additive manufacturing technique used to manufacture the screw 104 or the implant 100 include, directed energy deposition, stereolithography, selective laser sintering, direct metal laser sintering, electron beam melting, 3D printing, powder bed fusion, binder jetting, sheet lamination, material extrusion, material jetting, vat photopolymerization, mask-image-projection-based stereolithography, or any combination thereof. In some embodiments, a sacrificial material is used in combination with the metal material to result in a porous screw 104.

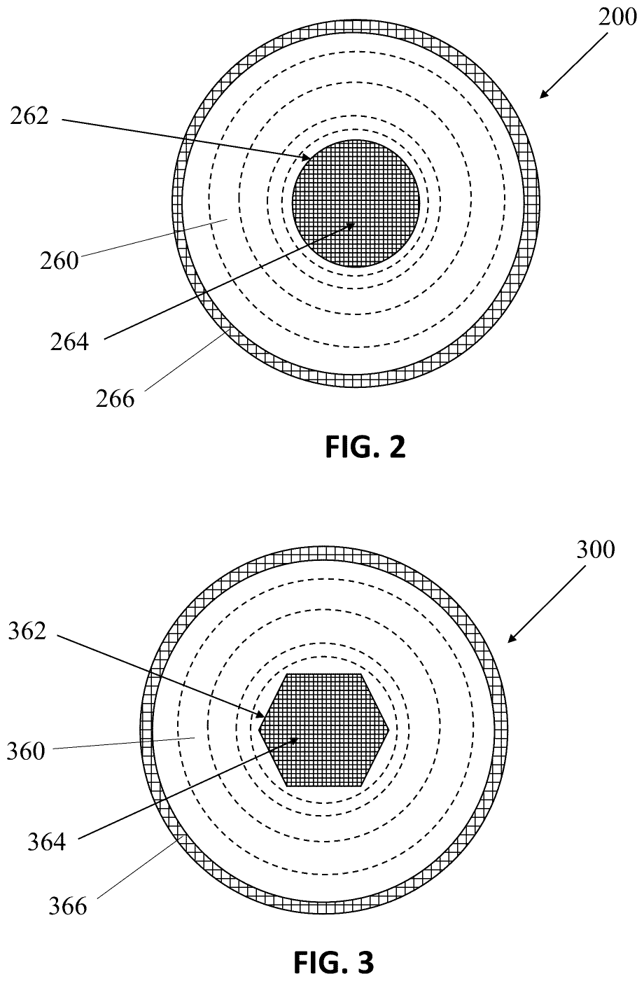

In some embodiments, the implant 100 extends from a proximal end 114 of the liner 102 to a distal end 112 of the screw 104. In some embodiments, the liner 102 has a convex semi-spherical shape having a concave liner surface (See, e.g., 260 and 360 in FIGS. 2 and 3) and a liner outer convex surface 116. In some embodiments, a first notch 118 is disposed into the liner outer convex surface 116 circumferentially about the longitudinal access. In some embodiments, a second notch 120 is disposed into the liner outer convex surface 116. In some embodiments one or more notches are disposed in the liner outer convex surface 116. In some embodiments, the one or more notches are disposed in successive layers along the liner outer convex surface 116.

In some embodiments, at least one indent 150 is disposed at least partially in the liner outer convex surface 116. In some embodiments, at least one indent 150 is disposed at least partially in the liner outer convex surface 116 and at least partially disposed in the first notch 118. In some embodiments, at least one indent 150 is disposed at least partially in the liner outer convex surface 116 and at least partially disposed in the second notch 120. In some embodiments, at least two of the indents 150 are arranged circumferentially around the first notch 118. In some embodiments, at least two of the indents 150 are arranged circumferentially around the first notch 118 and the second notch 120. In some embodiments, the indents 150 have a substantially semi-spherical interior surface. In some embodiments, the indents 150 have a concave interior surface. In some embodiments, the indents 150 have a convex surface. In some embodiments, the indents 150 have a combination of convex and concave surfaces. In some embodiments, the one or more notches and/or one or more indents help further secure the liner to the bone implant site compared to implants without said notched and/or implants. In some embodiments, the one or more notches and/or one or more indents are configured to receive and be at least partially filled with bone cement, which may be in addition to the screw portion 104 being threadably engaged with the bone, so as to further secure the implant 100 to the bone (e.g., glenoid bone).

In some embodiments, the liner outer convex surface 116 does not comprise any indents. In some embodiments, the liner outer convex surface 116 comprises a smooth profile without any layers. In some embodiments, the liner outer surface 116 comprises a layered configured configuration that tapers distally with a progressively decreasing diameter.

In some embodiments, the liner 102 has a thickness of about 4 mm. In some embodiments, the liner 102 comprises a polymer material. In some embodiments, the liner comprises a metal. In some embodiments, the liner 102 comprises polyethylene. In some embodiments, the liner 102 comprises an antioxidant. In some embodiments, the liner 102 comprises a polyethylene material infused with an antioxidant. In some embodiments, the antioxidant material may be homogeneously distributed throughout the polyethylene liner. In some embodiments, the antioxidant is coated on the surface of the polyethylene liner.

In some embodiments, the liner outer convex surface has a bevel 128 extending at least partially from the proximal end 114. In some embodiments, the implant 100 is configured to be disposed in a bone such that the proximal end 114 sits flush with the outer surface of an implant site (e.g., excision site) at the glenoid fossa. For example, the implant 100 may be configured to be inserted within the implant site at the glenoid, such that the entire liner, including the proximal end 114 is located at or below the outer surface of the bone surrounding the implant site. As disclosed herein, since the implant 100 were to sit flush with the surrounding bone, then less of the corresponding bone would need to be removed to allow for space for the implant 100 to interface with a corresponding humeral implant and/or humeral bone. For example, in a traditional shoulder system where the implant 100 is an inlay glenoid implant, it would be placed in the glenoid portion of the scapula bone, i.e., the glenoid fossa. In this example, less bone from the glenoid fossa, is removed to fit the inlay glenoid implant into the glenoid bone. As previously discussed, preserving native bone growth improves patient outcomes.

In some embodiments, the bone is a glenoid fossa. In some embodiments, the implant 100 is disposed at least partially within a bone. In some embodiments, the implant 100 is completely disposed in the bone such that the proximal surface 114 sits flush with the surrounding bone. In some embodiments, the implant 100 is secured without additional bone cement. In some embodiments, the implant 100 is secured with additional bone cement. In some embodiments, the implant 100 is configured to be secured at least partially in the glenoid fossa. In some embodiments, a portion of the glenoid fossa is not excised prior to the implant 100 being secured in the bone.

FIG. 2 illustrates an example of an implant 200 having a rim 266, a concave liner surface 260, and an inner portion 264. In some embodiments, the concave liner surface 260 extends in a distal direction from the rim 266. In some embodiments, the longitudinal axis of the inner portion 264 aligns with the longitudinal axis of the implant 200. In some embodiments, implant 200 is an inlay glenoid implant, and may correspond to implant 100 as described herein. For example, the implant may be disposed in the glenoid fossa region of the scapula bone such that the proximal end 114 is substantially flush with the surrounding bone outer surface. For example, when the implant 100 is fully disposed in an inlay position with a bone, the upper surface of the implant 100 may sit flush with the upper surface of the bone, wherein the outer edges of the rim 266 are flush with the surrounding bone.

In some embodiments, the inner portion edge 262 has a shape which is substantially triangular, square, rectangular, pentagonal, hexagonal (FIG. 3, heptagonal, octagonal, or combinations thereof. In some embodiments, the inner portion edge 262 has a substantially hexagonal shape. The inner portion 264 may extend in a distal direction from the inner portion edge to form a cavity. The inner portion 264 may have a distal inner portion surface (not shown) corresponding to the shape of the inner portion edge 262. The distal inner portion surface may be characterized as being located distally from the inner portion edge 262. In some embodiments, the inner portion 264 has interior surfaces in a generally cylindrical shape with an open top surface of the cylinder opening through the concave liner surface 260. In some embodiments, the inner portion 264 is configured to couple with a driver instrument to drive the implant 200 into a bone. For example, the inner portion may extend in a distal direction to form a cavity configured to couple with a driver instrument (not shown) to allow torque to be transmitted to the implant 200 to rotate the implant 200 such that one or more external screw (e.g., helical) threads 110 threadably engage and connect with the bone. In some embodiments, the bone is a glenoid fossa.

In some embodiments, the inner portion 264 may extend in a transverse direction from the inner portion edge 262 such that a surface of the inner portion 264 is substantially flush with the inner portion edge 262. In some embodiments, the inner portion 264 comprises the same materials as the screw 104. In some embodiments, the inner portion 264 protrudes in a proximal direction from the inner portion edge 262. In some embodiments, the inner portion 264 extends from the screw 104. In some embodiments, at least one of the inner portion 264, the screw 104, or the implant 200 is configured to couple with a baseplate (not shown). In some embodiments, the inner portion 264 is configured couple with and/or be received by channel disposed in a baseplate (not shown).

FIG. 3 illustrates an example of an implant 300 having a rim 366, a concave liner surface 360, and an inner portion 364. In some embodiments, the concave liner surface 360 extends in a distal direction from the rim 366. In some embodiments, the longitudinal axis of the inner portion 364 aligns with the longitudinal axis of the implant 300. In some embodiments, implant 300 is an inlay glenoid implant, and may correspond to implant 100 as described herein. In some embodiments, the implant 300 has an outer rim 366 which is located at a proximal end of the implant. In some embodiments, the inner concave liner surface 360 extends from the rim 366 in a distal direction. In some embodiments, the inner concave liner surface 360 extends in a distal direction towards an inner portion edge 362. In some embodiments, the inner portion 364 has a substantially hexagonal perimeter or shape. In some embodiments, the inner portion edge 362 opens into an inner portion 364. In some embodiments, the inner portion 364 extends in a distal direction from the inner concave liner surface 360 to form a cavity. In some embodiments, the inner portion 364 has interior surfaces forming a substantially hexagonal shape.

The inner portion 364 may have a distal inner portion surface (not shown) corresponding to the shape of the inner portion edge 362. The distal inner portion surface may be characterized as being located distally from the inner portion edge 362. In some embodiments, the inner portion 364 has interior surfaces in a substantially hexagonal prism shape with an open top surface of the prism opening through the concave liner surface 360. In some embodiments, the inner portion 364 is configured to couple with a driver instrument to drive the implant 300 into a bone. For example, the inner portion may extend in a distal direction to form a cavity configured to couple with a driver instrument (not shown) to allow torque to be transmitted to the implant 300 to rotate the implant 300 such that one or more external screw (e.g., helical) threads 110 threadably engage and connect with the bone. In some embodiments, the bone is a glenoid fossa.

In some embodiments, the inner portion 364 may extend in a transverse direction from the inner portion edge 362 such that a surface of the inner portion 364 is substantially flush with the inner portion edge 362. In some embodiments, the inner portion 364 comprises the same materials as the screw 104. In some embodiments, the inner portion 364 protrudes in a proximal direction from the inner portion edge 362. In some embodiments, the inner portion 364 extends from the screw 104. In some embodiments, at least one of the inner portion 364, the screw 104, or the implant 300 is configured to couple with a baseplate (not shown). In some embodiments, the inner portion 364 is configured couple with and/or be received by channel disposed in a baseplate (not shown).

In some embodiments, the inner portion 364 is configured to couple with a driver instrument to drive the implant 300 into a bone. In some embodiments, the bone is a glenoid fossa. In some embodiments, the implant 100 has a proximal surface corresponding to the proximal surface illustrated in implant 200. In some embodiments, the implant 100 has a proximal surface corresponding to the proximal surface illustrated in implant 300.

In some embodiments, the implant 400 and the screw 404 are detachably coupled. In some embodiments, the implant 400 and the screw 404 are a single component. In some embodiments, the implant 400 and the screw 404 are a single component configured to break apart into the implant 400 and the screw 404 when a force is applied. In some embodiments, the implant 400 has a substantially circular outer diameter. In some embodiments, the implant 400 is configured to threadably engage with a bone. According to one embodiment, the screw 404 may be secured to a bone, for example, using one or more external threads, ribs, protrusions, bone cement, barbs, grooves, or any other structure that enables the screw 404 to be secured to the bone.

In some embodiments, the screw 404 has a mantle portion 406 and pedestal portion 408. In some embodiments, the pedestal portion has at least one thread 410 disposed circumferentially around an outer surface of the pedestal 408. In some embodiments, the mantle portion 406 has a proximal surface having a concave coupling with and/or receiving at least a portion of the liner 402. In some embodiments, pedestal portion 408 extends from a convex, distal surface of the mantle portion 406. In some embodiments, the pedestal portion 108 extends from a center of the mantle portion 406 convex distal surface.

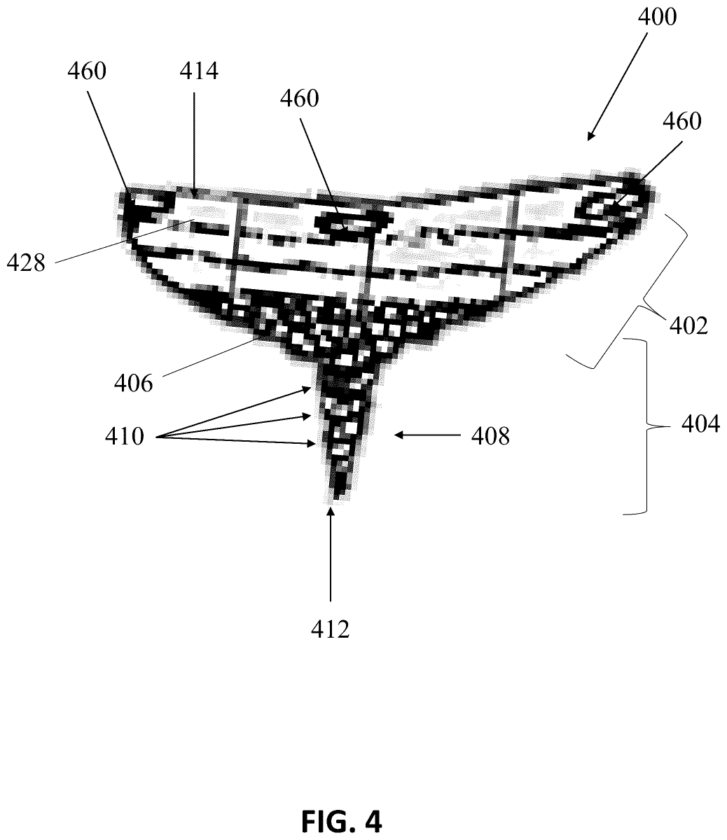

In some embodiments, for any implant described herein comprises one or more slots (e.g., reference character 460 in FIG. 4) disposed at least partially into the rim of an implant (e.g., reference character 428). In some embodiments, the one or more slots are configured to correspond to a grasping tool, a driving tool, or a combination thereof. FIG. 4 illustrates an example of an implant 400, which may be similar to any one of implant 100, 200, having a liner 402 and a screw 404, which may have a mantle portion 406 and a pedestal portion 408. In some embodiments, the pedestal portion has at least one thread 410 disposed circumferentially around an outer surface of the pedestal 408. In some embodiments, the rim 428 is configured with slots in the same configuration as the rim of a castellated nut. In some embodiments, the rim 428 is configured with slots 460 in the same configuration as the rim of a slotted nut. In some embodiments, the rim 428 is configured with slots 460 in a configuration of a rim of a castellated hex nut. In some embodiments, the rim 428 is configured with slots in a substantially similar configuration as the rim of a slotted hex nut. In some embodiments, one or more slots 460 are disposed in the outer surface of the liner 402 and/or the rim 428 or the liner 402. In some embodiments, one or more slots 460 are disposed in the outer surface of the liner 402 such that the rim 428 remains intact and a single surface. In some embodiments, one or more slots 460 are disposed from the outer surface of the liner 402 through to the concave inner surface 260, 360 (see FIGS. 2 and 3). In some embodiments, the implant 400 has no slots 460.

In some embodiments, slots 460 are substantially circular shape. In some embodiments, one or more slots are substantially oval in shape. In some embodiments, one or more slots have a shape which is substantially triangular, square, rectangular, pentagonal, hexagonal, heptagonal, octagonal, or combinations thereof. In some embodiments, the implant 400 has a single slot. In some embodiments, the implant 400 has two slots. In some embodiments, the implant 400 has six slots. In some embodiments, the implant 400 has any one of 1, 2, 3, 4, 5, 6, 7, 8, 9, 10, 11, 12, 13, 14, 15, 16, 17, 18, 19, 20, or more than 20 slots.

In some embodiments, one or more slots 460 are configured to couple with a driving instrument to drive the implant 400 into a bone. In some embodiments, one or more slots 460 are configured to couple with a grasping instrument to place the implant at and/or in the implant site.

In some embodiments, the implant 400 has a proximal surface corresponding to the proximal surface illustrated in implant 200. In some embodiments, the implant 400 has a proximal surface corresponding to the proximal surface illustrated in implant 300. In some embodiments, the implant 400 is placed in a glenoid fossa region of a scapula bone.

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined only by the preceding illustrative description.

In the appended claims, the terms “first,” “second,” “third,” and so forth, are used merely as labels and are not intended to impose numerical requirements on their objects.

The above specification and examples provide a complete description of the structure and use of illustrative configurations. Although certain configurations have been described above with a certain degree of particularity, or with reference to one or more individual configurations, those skilled in the art could make numerous alterations to the disclosed configurations without departing from the scope of this invention. As such, the various illustrative configurations of the methods and systems are not intended to be limited to the particular forms disclosed. Rather, they include all modifications and alternatives falling within the scope of the claims, and configurations other than the one shown may include some or all of the features of the depicted configurations. For example, elements may be omitted, modified, or combined as a unitary structure, connections may be substituted, or both.

Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one configuration or may relate to several configurations. Accordingly, no single implementation described herein should be construed as limiting and implementations of the disclosure may be suitably combined without departing from the teachings of the disclosure.

The previous description of the disclosed implementations is provided to enable a person skilled in the art to make or use the disclosed implementations. Various modifications to these implementations will be readily apparent to those skilled in the art, and the principles defined herein may be applied to other implementations without departing from the scope of the disclosure. Thus, the present disclosure is not intended to be limited to the implementations shown herein but is to be accorded the widest scope possible consistent with the principles and novel features as defined by the following claims. The claims are not intended to include, and should not be interpreted to include, means-plus- or step-plus-function limitations, unless such a limitation is explicitly recited in a given claim using the phrase(s) “means for” or “step for,” respectively.

What is claimed is:

1. A glenoid implant comprising:
a liner having a liner inner surface and a liner outer surface, the liner inner surface having a concave profile and configured to receive a corresponding convex surface of a humeral head or humeral implant; and
a screw having a mantle portion and a pedestal portion extending distally from the mantle portion, the mantle portion configured to be coupled with a distal portion of the liner outer surface such that the screw extends distally from the liner, the pedestal portion having one or more threads disposed thereabout configured to secure the implant to a glenoid fossa at an excision site thereof, such that the glenoid implant is configured to be disposed entirely or substantially entirely within the excision site, thereby having an inlay configuration.

2. The glenoid implant of claim 1, wherein one or both of the mantle portion and the pedestal portion comprise a porous material.

3. The glenoid implant of claim 2, wherein the porous material comprises an open cell porosity structure, a closed cell porosity structure, or a combination thereof.

4. The glenoid implant of claim 1, wherein the liner comprises an inner cavity portion about a distal portion of the liner inner surface.

5. The glenoid implant of claim 4, wherein the inner cavity portion has a generally cylindrical shape.

6. The glenoid implant of claim 4, wherein the inner cavity portion has a generally hexagonal prism shape.

7. The glenoid implant of claim 1, wherein the screw comprises a metal material.

8. The glenoid implant of claim 1, wherein the liner comprises a polyethylene material.

9. The glenoid implant of claim 8, further comprising an antioxidant material embedded in the polyethylene material.

10. The glenoid implant of claim 1, further comprising a textured interface disposed between the screw and the liner.

11. The glenoid implant of claim 10, wherein the textured interface has a checkerboard texture.

12. The glenoid implant of claim 1, further comprising at least one notch disposed in the liner outer surface.

13. The glenoid implant of claim 12, wherein the at least one notch includes a first notch and a second notch disposed in the liner outer surface.

14. The glenoid implant of claim 12, further comprising a plurality of indents disposed in the liner outer surface.

15. The glenoid implant of claim 14, wherein the plurality of indents have a generally concave shape.

16. The glenoid implant of claim 14, wherein the plurality of indents have a generally convex indent surface.

17. The glenoid implant of claim 14, wherein the plurality of indents are disposed at least partially in the at least one notch.

18. The glenoid implant of claim 14, wherein the plurality of indents are arranged radial-symmetrically about a longitudinal axis of the glenoid implant.

19. The glenoid implant of claim 14, wherein the indents are configured to retain bone cement to adhere the glenoid implant to the glenoid fossa.

20. The glenoid implant of claim 1, wherein the one or more threads comprise double helix threads.

21. The glenoid implant of claim 1, wherein the one or more threads is configured to secure the implant to a glenoid fossa through rotation therein.

22. The glenoid implant of claim 1, wherein the liner is configured to couple with the mantle portion of the screw through a threaded connection.

23. The glenoid implant of claim 21, wherein the liner is configured to decouple from the mantle portion of the screw through rotation.
